(19) United States
(12) Patent Application Publication
(10) Pub. No.: US 2004/0086422 A1
(43) Pub. Date: May 6, 2004

(54) Method and apparatus for destruction of biological matter in a ventilation system using UV radiation

(76) Inventors: Fred A. Elder, Webster, NY (US); Thomas K. Leach, Tonawanda, NY (US); Patrick M. Leach, East Aurora, NY (US); Timothy J. Leach, Orchard Park, NY (US); Donald Sniezak JR., Orchard Park, NY (US)

Correspondence Address: SIMPSON & SIMPSON, PLLC, 5555 MAIN STREET, WILLIAMSVILLE, NY, 14221-5406, US

(21) Appl. No.: 10/287,552

(22) Filed: Nov. 4, 2002

Related U.S. Application Data
(63) Continuation of application No. 10/286,286, filed on Nov. 1, 2002, now abandoned.

(51) Int. Cl. A61L 9/00; A61L 2/00; A62B 7/08; B01J 19/08

(52) U.S. Cl. 422/24; 422/1; 422/4; 422/5; 422/22; 422/121; 422/123; 422/186.3; 422/305; 422/306

(57) ABSTRACT

A method and apparatus for determining a number of UV generators needed to destroy at least 90% of biological matter in an air stream of a ventilation system is disclosed. The method comprises determining the number of UV generators needed to destroy a proportion of biological matter in the air stream of the ventilation system.

The computer program listing appendix is hereby expressly incorporated by reference in the present application.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### FIELD OF THE INVENTION

This invention relates to the destruction of biological matter. More specifically it relates to a method and apparatus for the destruction of biological matter using ultraviolet (UV) radiation. Even more specifically, the present invention relates to a method and apparatus for the destruction of biological matter by providing UV radiation in a ventilation system.

### BACKGROUND OF THE INVENTION

It is well known that UV emissions can destroy biological matter. This effect of UV emissions has been discussed in textbooks and periodicals such as: Wistreich, George A. and Lechtman, Max D., “Microbiology” 1988, Fifth edition, Macmillian Publishing Co., NY, N.Y. and Sharp, D. G., The Lethal Action of Short Ultraviolet Rays on Several Common Pathogenic Bacteria, J. Bact. 37, 447-459, 1939.

UV emitters have been used to destroy biological materials on the heating/cooling coils of heating, ventilation, and air conditioning (HVAC) systems, as shown in U.S. Pat. Nos. 5,817,276; 6,245,293; 6,267,924; 6,280,686; and 6,313,470. These patents disclose apparatuses that can destroy substantially all the biological matter on the heating/cooling coils and in the drain pans to prevent the biological matter from degrading the performance of the HVAC system. This makes the system more energy efficient. The apparatus incidentally destroys a portion of the biological matter in the air stream. However, the apparatus is insufficient to protect the building occupants from a terrorist attack, or any naturally occurring airborne disease-causing microorganism.

United States Patent Application No. 20020031460 discloses an apparatus that can be installed in a room to clean the air using UV lamps. Different sized lamps may be used to achieve different cleansing efficiencies. However, there is no disclosure of how to incorporate UV lamps into existing HVAC systems. The apparatus disclosed is a stand-alone unit. Further, there is no way to choose a custom level of cleansing efficiency for a plurality of specific biological agents. One can only install the limited number of fixed configurations and measure the effectiveness of these configurations.

Clearly, then, there is a longfelt need for a method and apparatus to destroy substantially all of the biological matter in the air stream of a ventilation system by determining the number of UV lamps needed to destroy a user determined proportion of biological agents.

### SUMMARY OF THE INVENTION

The present invention broadly comprises a method and apparatus for the destruction of biological matter in a ventilation system. The method comprises determining the number of UV generators needed to destroy biological matter in the air stream of the ventilation system.

A general object of the present invention is to provide a method and apparatus to destroy biological matter.

Another object of the present invention is to provide a method and apparatus for determining the number of UV lamps needed to destroy a user determined proportion of biological matter in the air stream of a ventilation system.

These and other objects, features and advantages of the present invention will become readily apparent to those having ordinary skill in the art upon a reading of the following detailed description of the invention in view of the drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1 is a top view of an HVAC duct showing UV generators proximate the heating/cooling coil downstream of the airflow;

FIG. 2 is a perspective view of an HVAC duct having UV generators installed according to the method of the present invention;

FIG. 3 is a view of the kill zone parameters; V

FIG. 4 is a screen display illustrating the menu for selecting a new or existing customer;

FIG. 5 is a screen display illustrating an addition of a new customer to the database;

FIG. 6 is a screen display illustrating a completed database entry for a customer;

FIG. 7 is a screen display illustrating a selection of an existing customer;

FIG. 8 is a screen display illustrating the determination of the number of bulbs needed for a given installation;

FIG. 9 is a screen display illustrating the entry of the physical characteristics of the installation;

FIG. 10 is a screen display illustrating the entry of a new organism to the database; and,

FIG. 11 is a screen display illustrating changing a program constant.

### DETAILED DESCRIPTION OF THE PREFERRED EMBODIMENT

It should be appreciated that, in the detailed description of the invention which follows, like reference numbers on different drawing views are intended to identify identical structural elements of the invention in the respective views. In the present specification and claims, the term “ventilation system” is intended to mean any apparatus for circulating air in a building.

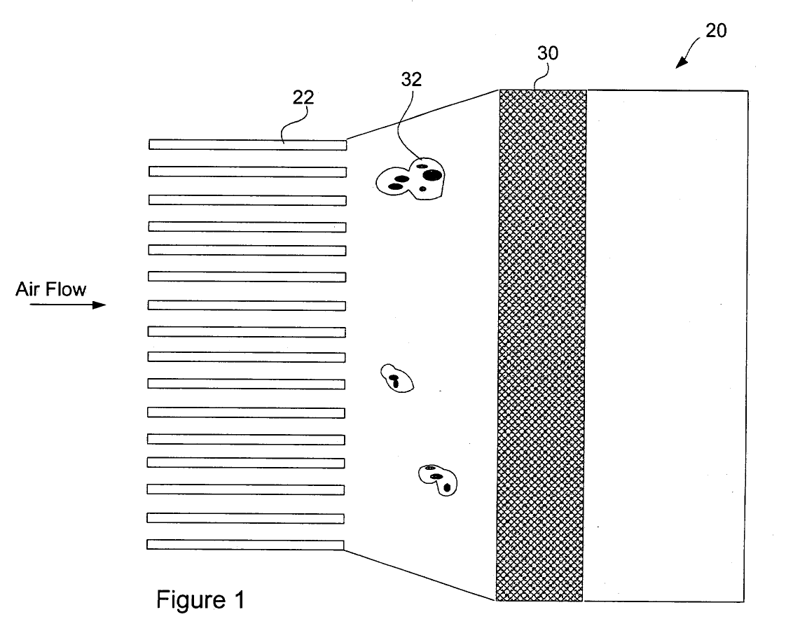

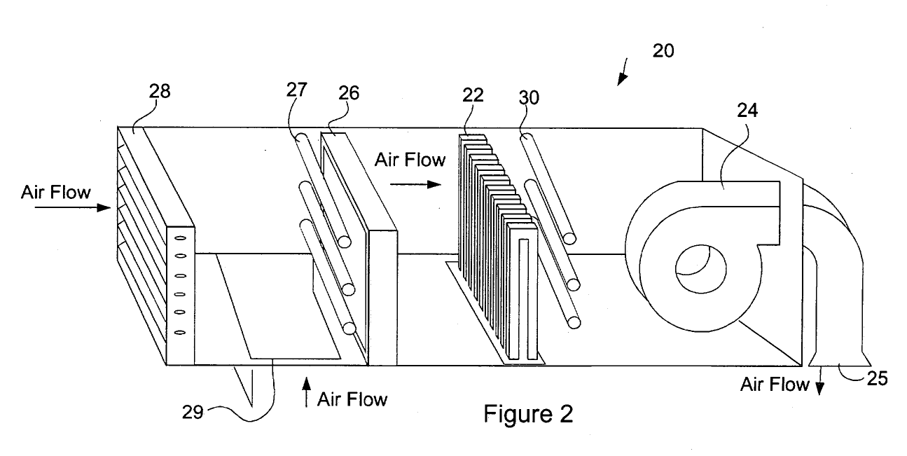

FIG. 1 illustrates a top view of an HVAC duct 20 with UV lamps 30 installed therein according to the present invention. Lamps 30 are installed downstream from the heating/cooling coils 22 to kill biological agents 32. As shown in FIG. 2, fan 24 circulates the air through duct 20. Filter 26, located upstream of coil 22 in FIG. 2, removes a portion of the airborne particles entering duct 20. Lamps 27 are used to kill any biological agents lodged in filter 26. Air can enter duct 20 from inside the building through return air passage 29. Air can enter duct 20 from the outside through damper 28. Sterilized air is supplied to the building through exit passage 25.

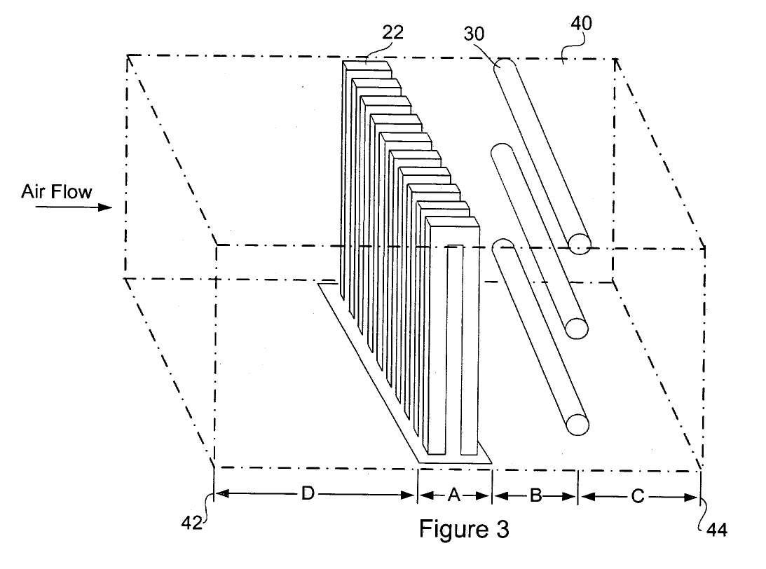

In a preferred embodiment, UV lamps 30 are cylindrical, and emit UV radiation in all directions. This kills biological agents 32 both upstream and downstream from the lamps, creating kill zone 40, shown in FIG. 3. Kill zone 40 extends the width and height of the duct 20 and a length from a location 42 upstream of the coil 22 to a location 44 downstream of lamps 30. The present invention creates a kill zone of sufficient size and intensity to kill a user specified proportion of the biological agents in the air stream of the HVAC duct.

In a preferred embodiment, lamps of various lengths emitting 253.7 nanometer ultraviolet radiation made by Steril-Aire, U.S.A. Inc. of Cerritos, Calif. are used. However, it should be readily apparent to one skilled in the art that other UV lamps may be used, and these modifications are intended to be within the spirit and scope of the invention as claimed. Also in a preferred embodiment, the method of the invention is practiced by a software program, for example the program disclosed on the compact disc included in the present specification. However, it should be readily apparent to one skilled in the art that a software program is not necessary to practice the present invention, and these modifications are within the spirit and scope of the invention as claimed.

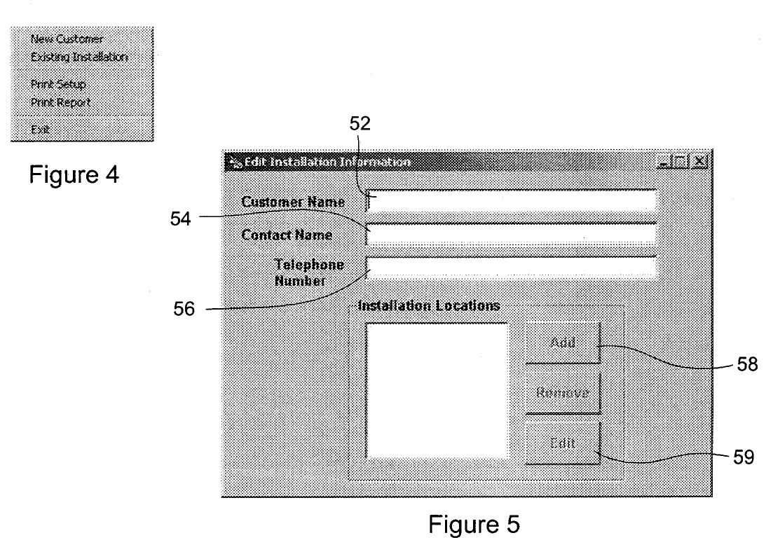

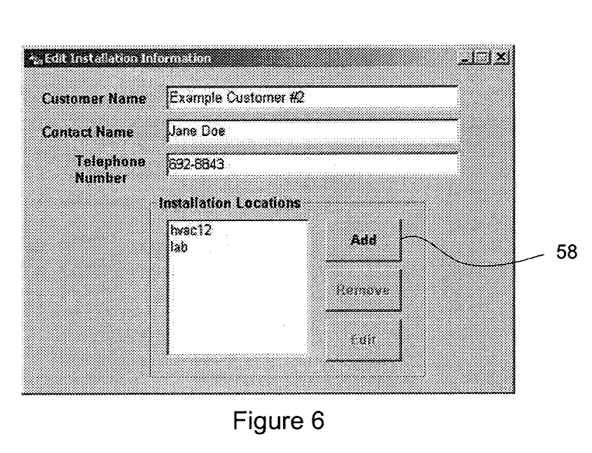

In the software embodiment of the present invention disclosed herein, the first step in practicing the invention is to create a new customer in the database or select an existing customer, as illustrated in FIG. 4. If the entry of a new customer is selected, the software creates the window shown in FIG. 5. The user enters the name of the customer in box 52, the customer contact person in box 54, and the contact's telephone number in box 56. The new customer is the entity having UV lamps installed in the HVAC system of its building according to the present invention. When all of the above-listed information is entered, control button 58 can then be clicked to add an installation, as shown in FIG. 6.

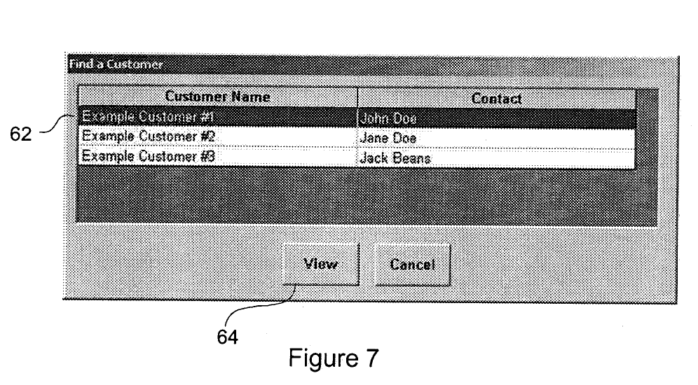

If an existing customer is to be edited, “Existing Installation” is chosen from the menu shown in FIG. 4. The software then creates the window shown in FIG. 7. Existing customer 62 is highlighted, and then control button 64 is clicked to view that customer. The window illustrated in FIG. 6 is then created containing the information for existing customer 62.

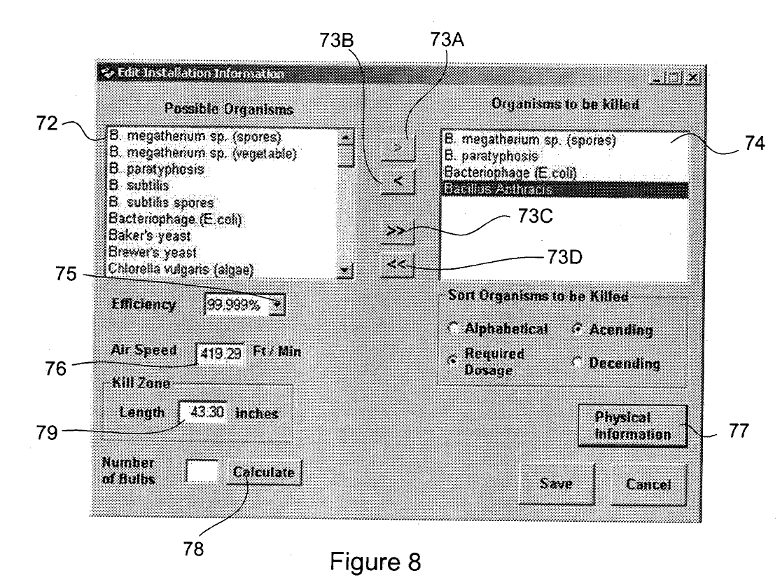

After an installation is added using control button 58, the parameters of the HVAC system can be entered by clicking “Edit” control button 59. This brings up the screen shown in FIG. 8. Biological agents existing in the database are shown in box 72. Biological organisms to be destroyed are shown in box 74. Control buttons 73A, 73B, 73C, and 73D are clicked when organisms are highlighted to add or remove organisms from box 74. An efficiency level is chosen using control button 75. This is the proportion of biological agent to be destroyed by the UV lamps. For example, if the user chooses Bacillus anthracis as an agent to be destroyed, the user can choose efficiencies of 90%, 99%, 99.9%, 99.99%, 99.999%, 99.9999%, and 99.99999%. Each efficiency level corresponds to a dosage level for killing that proportion of the agent. Thus, a user is only limited to the dosages, efficiencies, and agents entered into the database. It should be readily apparent to one skilled in the art that any biological agent may be destroyed in this manner to any level of efficiency, and these modifications are intended to be within the spirit and scope of the invention as claimed. As discussed below, additional organisms may be entered into the database.

When multiple organisms are entered into box 74, the highest dosage required by any of the organisms for the efficiency level chosen for that organism is used for the computation shown below. It should be readily apparent to one skilled in the art that if a higher dosage level is used than that needed for the efficiency level chosen, then the organism will be killed at an even higher efficiency level.

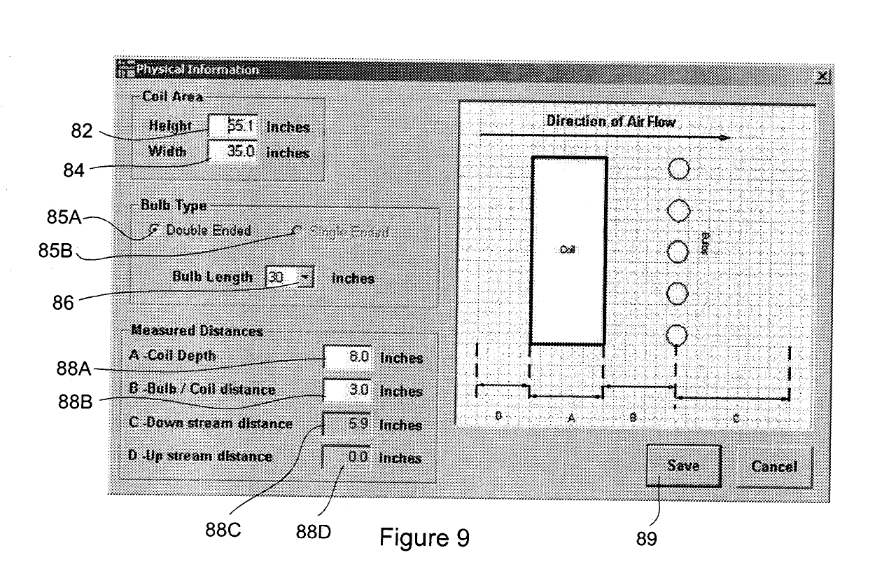

The air speed in the duct is entered into box 76. The effective kill zone length is contained in box 79. This is computed from the parameters entered into the “Physical Information” screen, shown in FIG. 9. To enter these parameters, “Physical Information” control button 77 is clicked.

The following parameters are entered on the “Physical Information” screen: coil height in box 82, coil width in box 84, bulb type with selection circles 85A and 85B, bulb length with control button 86, coil depth in box 88A, and bulb to coil distance in box 88B. In the disclosed embodiment, the upstream distance is fixed at 0 in box 88C and the downstream distance is fixed at 5.9 inches in box 88D. When the “Physical Information” screen is exited by clicking “Save” control button 89, the effective Kill Zone length is then computed according to the following formula and entered into Kill Zone box 79 on the “Edit Installation” screen:

Effective Kill Zone Length=Upstream Distance+4*Coil Depth+Bulb to Coil Distance+Downstream Distance

where Upstream Distance is the distance labeled D on FIG. 3, Coil Depth is the distance labeled A on FIG. 3, Bulb to Coil Distance is the distance labeled B on FIG. 3, and Downstream Distance is the distanced labeled C on FIG. 3.

Thus, the physical length of Kill Zone 40 is different from the effective kill zone length because the effective kill zone length includes four times the coil depth. This is due to the interaction of the agents with the coil. The interaction causes the agents to remain in the coil as if the coil was four times its actual depth.

When all of the parameters of the HVAC system are entered into the appropriate spaces on the “Edit Installation” and “Physical Information” screens, the number of lamps needed to destroy the biological agents chosen to the efficiency desired is computed by clicking “Calculate” control button 78. The number of bulbs is computed according to the following formula:

$${N}_{B} = \frac{D \times AS}{{L}_{KZE} \times {RF}^{2} \times IF \times {N}_{AL} \times {I}_{AL}}$$

where N_{B} is the number of bulbs, D is the highest dosage of all the dosages corresponding to the each of the organisms in box 74 at each chosen efficiency level, AS is the air speed, L_{KZE} is the Effective Kill Zone Length computed above, RF is the reflectance factor of the coil (a physical constant set to 1.5 in the disclosed embodiment), N_{AL} is the number of arc lengths (N_{AL}=(1.02*Bulb Length)−5.44), I_{AL} is the intensity per unit arc length (I_{AL}=5.52 in the disclosed embodiment), and IF is the intensity factor computed as follows:

| | |
|---|---|
| IF = (691.59/Coil to Bulb Distance) | if Coil to Bulb Distance ≧ 3 |
| | inches OR |
| IF = 230.53 | if Coil to Bulb Distance < 3 |
| | inches. |

As shown in the drawings, the dimensions of the duct, coil, and bulb are entered in inches. The air speed is entered in feet/minute. The dosages in the enclosed database are in microwatt-seconds/square centimeter. The constants in the enclosed software are calculated to change the entered English measurement units to Metric units, and perform the calculations with Metric units. However, it should be readily apparent to one skilled in the art that any system of measurement may be used for entering the data and performing the calculations, and these modifications are within the spirit and scope of the invention as claimed.

The enclosed database contains dosage levels required to kill a plurality of microorganisms at varying efficiencies. However, it should be readily apparent to one skilled in the art that the present invention can be used to kill any known biological matter, such as but not limited to: Yersinia pestis, variola major, Francisella tularensis, viral hemorrhagic fevers such as filoviruses (e.g. Ebola, Marburg) and arenaviruses (e.g. Lassa, Machupo), Brucella species, Clostridium perfringens, Salmonella species, Escherichia coli O157:H7, Shigella, Burkholderia mallei, Burkholderia pseudomallei, Chlamydia psittaci, Coxiella burnetti, Ricin toxin from Ricinus communis, Staphylococcal enterotoxin B, Rickettsia prowazekii, viral encephalitis such as alphaviruses (e.g. Venezuelan equine encephalitis, eastern equine encephalitis, western equine encephalitis), Vibrio cholerae, Cryptosporidium parvum, Nipah virus, and hantavirus.

The above-computed number of UV lamps having the length and style entered into the window shown in FIG. 9 are installed in the duct at the bulb to coil distance entered box 88B. Lamps 30 destroy all of the biological agents entered into box 74 at the efficiency level chosen with control button 75.

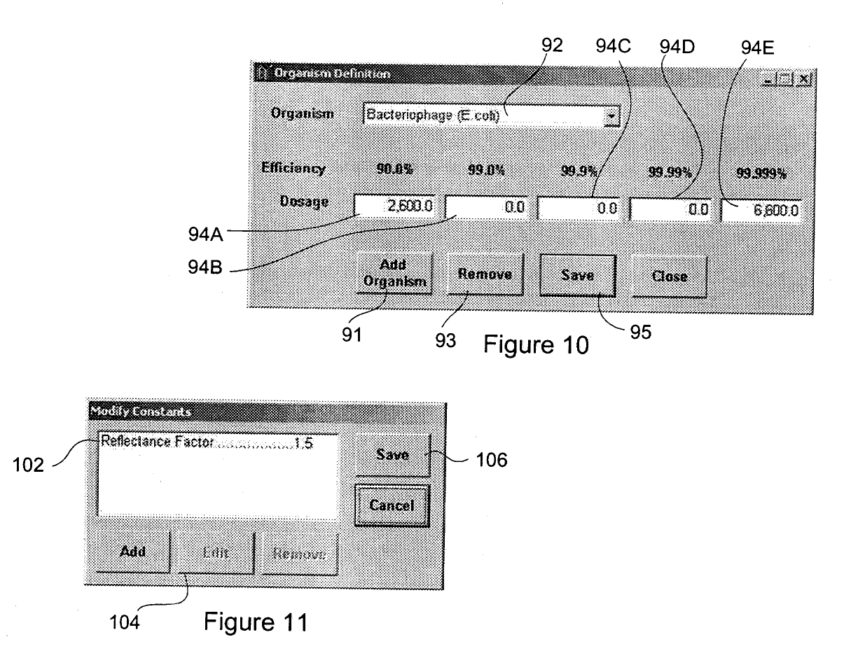

An example of the entry of a new organism to the database is illustrated in FIG. 10. The user enters a new biological agent by clicking control button 91. The name of the organism is entered into box 92. The dosages required to kill the newly entered organism at the efficiencies 90.0%, 99.0%, 99.9%, 99.99%, and 99.999% are entered into boxes 94A, 94B, 94C, 94D, and 94E, respectively. If the dosage for an efficiency level is left at 0, that efficiency level is not available when control button 75 is clicked. For the example shown in FIG. 10, 94A and 94E contain non-zero values, and 94B, 94C, and 94D contain zero values. Thus, with this example data saved, the only efficiency values that can be chosen with control button 75 for Bacteriophage (E. coli) are 90.0% and 99.999%. An organism may be deleted by clicking control button 93. To save an entered organism, control button 95 is clicked. FIG. 10 shows the entry of dosage levels for efficiency levels of 90.0%, 99.0%, 99.9%, 99.99%, and 99.999%. It should be readily apparent to one skilled in the art that efficiency levels and dosages corresponding to any desired efficiency levels can be used, and these modifications are intended to be within the spirit and scope of the invention as claimed.

The physical constants used in the program may also be modified, as shown in FIG. 11. The only physical constant in the disclosed embodiment is reflectance factor 102. To change the value of the reflectance factor, reflectance factor 102 is highlighted and edit button 104 is clicked. The newly entered value is saved by clicking control button 106.

Thus, it is seen that the objects of the present invention are efficiently obtained, although modifications and changes to the invention should be readily apparent to those having ordinary skill in the art, and these modifications are intended to be within the spirit and scope of the invention as claimed.

What is claimed is:

1. A method for destroying a predetermined percentage of biological matter in an air stream of a ventilation system comprising determining a number of ultraviolet (UV) generators to be placed within said ventilation system, said number determined according to a formula, wherein said formula calculates said number of UV generators based on at least one of the following factors: identity of said biological matter to be destroyed, said predetermined percentage, a dosage level of UV radiation, air speed in a duct of said ventilation system, a linear dimension of said duct, a length of said generators, and a distance between said generators and a coil of said duct.

2. The method recited in claim 1 wherein said formula is:
$${N}_{G} = \frac{D \times AS}{{L}_{KZE} \times {RF}^{2} \times IF \times {N}_{AL} \times {I}_{AL}}$$
where N_{G} is said number of generators, D is a dosage corresponding to said percentage of said biological matter to be destroyed, AS is an air speed, L_{KZE} is an Effective Kill Zone Length=Upstream Distance+4*Coil Depth+Generator to Coil Distance+Downstream Distance, RF=1.5, N_{AL}=(1.02*Generator Length)−5.44, I_{AL}=5.52, and IF is an intensity factor computed as follows:

| | |
|---|---|
| IF = (691.59/Coil to | if Coil to Generator Distance ≧ 3 inches OR |
| Generator Distance) | |
| IF = 230.53 | if Coil to Generator Distance < 3 inches. |

3. The method recited in claim 1 wherein said predetermined percentage is selected from the group consisting of: 90%, 99%, 99.9%, 99.99%, 99.999%, 99.9999%, and 99.99999%.

4. The method recited in claim 1 wherein said biological matter comprises at least one of the following: Bacillus anthracis, B. megatherium sp. (spores), B. megatherium sp. (vegetable), B. paratyphosis, B. subtilis, B. subtilis spores, Bacteriophage (E. coli), Baker's yeast, Brewer's yeast, Chlorella vulgaris (algae), Clostridium tetan, Common yeast cake, Corynebacterium diptheria, Eberthelia typosa, Escherichilla coli, Infectious Hepatitis, Influenza, Leptospira (infectious jaundice), Micrococcus candidus, Micrococcus sphaeroides, Mycibaterium tuberculosis, Neisseria catarrhalis, Nematode eggs, Paramecium, Penicilium roqueforti (green), Phisopus nigricans (black), Phytomonas tumefaciens, Poliomyelitis (Poliovirus), Proteus vulgaris, Pseudomonas aeruginosa, Pseudomonas fluorescens, S. enteritidis, S. typhimurium, Saccharomyces sp, Saccharomyces carevisiae, Saccharomyces ellipsoideus, Salmonella paratyphi (enteric fever), Salmonella Typhosa (typhoid fever), Sarcina lutea, Serratia marcescens, Shigella flexneri (dysentery), Shigella dysenteriea (dysentery), Shigella paradysenteriae, Spirflum rubrum, Staphylococcus albus, Steptococcus hemolyticus, Streptococcus lactis, Streptococcus viridans, Tobacco mosaic, Vibria cholerse (cholera), Yersinia pestis, variola major, Francisella tularensis, viral hemorrhagic fevers, Brucella species, Clostridium perfringens, Salmonella species, Escherichia coli O157:H7, Shigella, Burkholderia mallei, Burkholderia pseudomallei, Chlamydia psittaci, Coxiella burnetti, Ricin toxin from Ricinus communis, Staphylococcal enterotoxin B, Rickettsia prowazekii, viral encephalitis, Vibrio cholerae, Cryptosporidium parvum, Nipah virus, and hantavirus.

5. The method recited in claim 4 wherein said viral hemorrhagic fever is a filovirus.

6. The method recited in claim 5 wherein said filovirus is Ebola.

7. The method recited in claim 5 wherein said filovirus is Marburg.

8. The method recited in claim 4 wherein said viral hemorrhagic fever is an arenavirus.

9. The method recited in claim 8 wherein said arenavirus is Lassa.

10. The method recited in claim 8 wherein said arenavirus is Machupo.

11. The method recited in claim 4 wherein said viral encephalitis is an alphavirus.

12. The method recited in claim 5 wherein said alphavirus is Venezuelan equine encephalitis.

13. The method recited in claim 5 wherein said alphavirus is eastern equine encephalitis.

14. The method recited in claim 5 wherein said alphavirus is western equine encephalitis.

15. A method for the destruction of a predetermined percentage of biological matter in an air stream of a ventilation system comprising installing a number of UV generators in said ventilation system wherein said number is determined according to a formula, wherein said formula calculates said number of UV generators based on at least one of the following factors: identity of said biological matter to be destroyed, said predetermined percentage, a dosage level of UV radiation, air speed in a duct of said ventilation system, a linear dimension of said duct, a length of said generators, and a distance between said generators and a coil of said duct.

16. The method recited in claim 15 wherein said formula is:
$${N}_{G} = \frac{D \times AS}{{L}_{KZE} \times {RF}^{2} \times IF \times {N}_{AL} \times {I}_{AL}}$$
where N_{G} is said number of generators, D is a dosage corresponding to said percentage of said biological matter to be destroyed, AS is an air speed, L_{KZE} is an Effective Kill Zone Length=Upstream Distance+4*Coil Depth+Generator to Coil Distance+Downstream Distance, RF=1.5, N_{AL}=(1.02*Generator Length)−5.44, I_{AL}=5.52, and IF is an intensity factor computed as follows:

| | |
|---|---|
| IF = (691.59/Coil to | if Coil to Generator Distance ≧ 3 inches OR |
| Generator Distance) | |
| IF = 230.53 | if Coil to Generator Distance < 3 inches. |

17. An apparatus for determining a number of UV generators needed to destroy a predetermined percentage of biological matter in a ventilation system comprising:
means for determining a number of UV generators to be placed within said ventilation system, said number determined according to a formula, wherein said formula calculates said number of UV generators based on at least one of the following factors: identity of said biological matter to be destroyed, said predetermined percentage, a dosage level of UV radiation, air speed in a duct of said ventilation system, a linear dimension of said duct, a length of said generators, and a distance between said generators and a coil of said duct.

18. The apparatus recited in claim 17 wherein said means for determining a number of UV generators to be placed within said ventilation system comprises a general purpose computer specially programmed to determine said number of UV generators.

19. The apparatus recited in claim 18 wherein said computer is programmed with the following equation:
$${N}_{G} = \frac{D \times AS}{{L}_{KZE} \times {RF}^{2} \times IF \times {N}_{AL} \times {I}_{AL}}$$
where N_{G} is said number of generators, D is a dosage corresponding to said percentage of said biological matter to be destroyed, AS is an air speed, L_{KZE} is an Effective Kill Zone Length=Upstream Distance+4*Coil Depth+Generator to Coil Distance+Downstream Distance, RF=1.5, N_{AL}=(1.02*Generator Length)−5.44, I_{AL}=5.52, and IF is an intensity factor computed as follows:

| | |
|---|---|
| IF = (691.59/Coil to | if Coil to Generator Distance ≧ 3 inches OR |
| Generator Distance) | |
| IF = 230.53 | if Coil to Generator Distance < 3 inches. |
